SEQUENCE LISTING (18 sequences)

SEQ ID NO: 1 - DNA, 24 bp, Artificial Sequence
  primer_bind (1)...(24): Reverse hphF
agcggataacaatttcacacagga

SEQ ID NO: 2 - DNA, 25 bp, Artificial Sequence
  primer_bind (1)...(25): hphF
tcgcggtgagttcagatcttttcat

SEQ ID NO: 3 - DNA, 20 bp, Artificial Sequence
  primer_bind (1)...(20): gGFP-Cla1
ccatcgatatggcgagcaag

SEQ ID NO: 4 - DNA, 21 bp, Artificial Sequence
  primer_bind (1)...(21): reverse gGFP-Cla1
ccgaattctcatgtttgacag

SEQ ID NO: 5 - DNA, 24 bp, Artificial Sequence
  primer_bind (1)...(24): HEWL-Cla1
ggatcgatgaggtctttgctaatc

SEQ ID NO: 6 - DNA, 24 bp, Artificial Sequence
  primer_bind (1)...(24): reverse HEWL-EcoR1
gggaattctcacagccggcagcct

SEQ ID NO: 7 - DNA, 25 bp, Artificial Sequence
  primer_bind (1)...(25): PDGF-Cla1
gtttatcgataagaaggagatatac

SEQ ID NO: 8 - DNA, 25 bp, Artificial Sequence
  primer_bind (1)...(25): reverse PDGF-EcoR1
gttagcagccgaattcctattaggt

SEQ ID NO: 9 - DNA, 24 bp, Artificial Sequence
  primer_bind (1)...(24): PBS
caagcgcgcaattaaccctcacta

SEQ ID NO: 10 - DNA, 22 bp, Artificial Sequence
  primer_bind (1)...(22): PBS terminator
caccacctactcacgactgttg

SEQ ID NO: 11 - DNA, 23 bp, Artificial Sequence
  primer_bind (1)...(23): Kpn1-pro-gGFP
gagtggtaccagatgtgagtcgt

SEQ ID NO: 12 - DNA, 22 bp, Artificial Sequence
  primer_bind (1)...(22): Sac1-ter-gGFP
ggagctcgataaccgggatccg

SEQ ID NO: 13 - DNA, 26 bp, Artificial Sequence
  primer_bind (1)...(26): HSP70-100
gcctcgagatttgttccaagttcaaa

SEQ ID NO: 14 - DNA, 26 bp, Artificial Sequence
  primer_bind (1)...(26): HSP70-200
agctcgaggaagaagaacgtggtaac

SEQ ID NO: 15 - DNA, 26 bp, Artificial Sequence
  primer_bind (1)...(26): HSP70-300
atctcgagggctaaaggaagcgagac

SEQ ID NO: 16 - DNA, 26 bp, Artificial Sequence
  primer_bind (1)...(26): HSP70-400
atctcgagttgcgtcagccttgtacc

SEQ ID NO: 17 - DNA, 26 bp, Artificial Sequence
  primer_bind (1)...(26): HSP70-500
atctcgagatgacacagcacaccaag

SEQ ID NO: 18 - DNA, 29 bp, Artificial Sequence
  protein_bind (1)...(29): reverse antiptro-all
atccatggcctccgcgaccggctgcagaa

(19) United States
(12) Patent Application Publication
(10) Pub. No.: US 2003/0232413 A1
(43) Pub. Date: Dec. 18, 2003

(54) Transformed fungi for production of recombinant proteins

(75) Inventors: Richard Belanger, Cap-Rouge (CA); Caroline Labbe, Levis (CA); Yali Cheng, Sainte-Foy (CA)

Correspondence Address: OGILVY RENAULT, 1981 MCGILL COLLEGE AVENUE, SUITE 1600, MONTREAL, QC, H3A2Y3, CA

(73) Assignee: UNIVERSITE LAVAL, Quebec (CA)

(21) Appl. No.: 10/443,976

(22) Filed: May 23, 2003

Related U.S. Application Data
(60) Provisional application No. 60/382,584, filed on May 24, 2002.

(51) Int. Cl. C12P 21/02; C12N 1/18; C12N 15/74

(52) U.S. Cl. 435/69.1; 435/254.2; 435/320.1; 435/483

(57) ABSTRACT

The present invention relates to the use of Pseudozyma spp. for the production of recombinant polypeptides, proteins or peptides, by genetically transforming fungi with a plasmids or appropriate DNA expression vector.

### BACKGROUND OF THE INVENTION

i) Field of the Invention

The invention relates to the use of fungi as a host in a host-vector system and method for the production of recombinant products. The method and system of the present invention pertain particularly to means offering the potential of easy scale adaptability for the production of different recombinant polypeptides. Also, the present invention allows for the production of recombinant polypeptides with more complex configuration properties than what is possible in systems using other types of microorganism.

ii) Description of Prior Art

Gene manipulation technologies have made it possible to produce useful proteins and other recombinant products (peptides, polypeptides, and the like) in large amounts with bacteria, yeasts and fungi, animal cell cultures (human, mammal, and insect cells), or with transgenic plants and animals.

From an industrial point of view, an ideal host system should enable the production of recombinant products that are safe, easy to purify, have biological activities identical to that of the natural protein with high yields and at an economically attractive cost. Although all the aforementioned host systems have the (potential) ability to produce recombinant products, these systems are not completely satisfactory for the efficient production of the vast diversity of recombinant products. For example, a protein natively expressed within the endoplasmic reticulum membrane can be expressed extensively in bacteria but will be folded incorrectly, since bacteria lack the cellular apparatus necessary to carry out folding. Therefore, proteins requiring folding will have no or much less activity when produced in bacteria and will require additional treatment to fold the proteins. Conversely, an eukaryote protein can be expressed in the proper organelle of a foreign insect or mammal cell, but could be rendered useless due to low yield and the undue cost associated with these technologies.

An alternative to these technologies is the expression of exogenous proteins within fungi, which are eukaryote cells and multiply at an extensive rate. Fungi secrete substantial amounts of protein, notably hydrolytic enzymes. Pseudozyma spp., which are basidiomycetous fungi, are members of the most evolved class of fungi and are phylogenetically closer to animal cell than any other class of organisms. This indicates that they would synthesize more complex proteins than lower fungi such as the yeasts Saccharomyces and Pichia or bacteria such as E. coli and Streptomyces spp. However, very little is known in terms of genetics and physiology of Pseudozyma spp., with the exception of phylogeny. For example, some species of Pseudozyma are known to produce native enzymatic proteins of industrial importance.

PCT application WO01/96536 describes methods for producing native aspartic acid proteases from Pseudozyma sp.

U.S. Pat. No. 6,352,841 describes native lipase B from Pseudozyma antarctica (also known as Candida antarctica).

Over the past few years, genetic transformation systems have been successfully developed for many organisms, including fungi. However, Pseudozyma spp. appeared neglected since neither methods to perform genetic transformation nor production of recombinant products within these organisms are known at this time.

Considering the state of the art mentioned above, there is still a vast place in which to provide new and alternative living systems and methods for the production of recombinant polypeptides.

### SUMMARY OF THE INVENTION

One object of the present invention is to provide the use of a Pseudozyma spp. fungal strain as a host in a host-vector system for the production of recombinant proteins and other recombinant products.

Another object of the present invention is to provide a method for the production of a recombinant protein in a strain of Pseudozyma comprising the steps of transforming a strain of Pseudozyma, which can be performed through transformation of protoplasts, with an expression vector, and cultivating the transformed strain under conditions allowing synthesis of a recombinant product from the expression vector.

Someone skilled in the art will recognize that transformation of protoplasts will facilitate the preparation of fungi containing the expression vector, for the production of the recombinant polypeptides of interest.

In accordance with the present invention there is provided a method for producing recombinant proteins wherein the strain of Pseudozyma utilized to produce such proteins may be Pseudozyma antarctica, Pseudozyma aphidis, Pseudozyma flocculosa, Pseudozyma fusiformata, Pseudozyma prolifica, Pseudozyma rugulosa, Pseudozyma tsukubaensis or any fungal strain which can be identified as a member of the Pseudozyma genus based on conventional and/or molecular identification techniques.

The expression vector used in the present invention may be a plasmid, an expression cassette, or a virus, and may comprise a DNA sequence encoding a recombinant protein or peptide, and a promoter functionally active in the genus Pseudozyma. The vector may also comprise a target sequence that allows the secretion of the expressed recombinant product to the correct organelle or outside the limits of the cell.

The promoter that drives the production of the recombinant product in the expression vector can be any promoter that is functional in Pseudozyma spp. These promoters include native promoters, i.e. promoters that are naturally found in the genome of the fungal genus, as well as exogenous promoters. The promoter can constitutively drive the expression of the gene under its control or can be an inducible promoter.

Another object of the present invention is to provide a fungus of the genus Pseudozyma genetically transformed for producing a recombinant protein or peptide.

For the purpose of the present invention the following terms are defined below.

The terms “fungus” and “fungi” as used herein are intended to mean single- or multiple-cell organisms that lack chlorophyll and have cell walls that contain chitin, cellulose or both. It is understood that fungi include molds and yeasts.

The term “gene” as used herein is intended to mean a DNA sequence responsible for the production of a protein (or polypeptide) or a functional fragment thereof. This includes first and foremost the actual coding sequence, which dictates the specific order of amino acids in a polypeptide.

The terms “recombinant protein” and “recombinant product” are intended to mean a protein to be expressed from a nucleic acid molecule which contains at least two parts that originate from at least two different kind of nucleic acid molecules that have been joined to form a single nucleic acid molecule. “Recombinant protein” and “recombinant product”, as used herein are intended to mean both heterologous and homologous (endogenous) proteins, as defined below.

The expressions “heterologous polypeptides” and “heterologous product”, as used herein are intended to mean any protein, peptide, polypeptide or the like that is not native to the fungal host of the present invention. A heterologous gene encoding for the heterologous protein may therefore originate from animal, including human, plant, fungal, bacterial or any other living species. In addition, the heterologous gene can be a synthetic gene, synthesized exclusively by human hand or naturally produced and further modified by said human hand, where the heterologous product is different than what is naturally found in the host cell. A recombinant polypeptide may therefore originate from the fungus itself (homologous polypeptide) although having been genetically manipulated for production purposes.

The term “promoter” as used herein is intended to mean non-coding regulatory sequences for transcription, usually located nearby the start of the coding sequence, which may be referred to as the gene promoter or the regulatory sequence. Put into a simplistic yet basically correct way, it is the interplay of the promoter with various specialized proteins called transcription factors that determine whether or not a given coding sequence may be transcribed and eventually translated into the actual protein encoded by the gene.

The term “vector” as used herein refers to a nucleic acid sequence, e.g., DNA derived from a plasmid, cosmid, virus, or synthesized by chemical or enzymatic means, into which one or more fragments of nucleic acid may be inserted or cloned, where the nucleic which encode for particular genes. The vector can contain one or more unique restriction sites for this purpose, and may be capable of autonomous replication in a defined host or organism such that the cloned sequence is reproduced. The vector may have a linear, circular, or supercoiled configuration and may be complexed with other vectors or other material for certain purposes. The components of a vector can contain but is not limited to a DNA molecule incorporating DNA; a sequence encoding an excision protein or another desired product; and regulatory elements for transcription, translation, RNA stability, and replication.

The term “polypeptide” as used herein, refers to any amino acid sequence, oligopeptide, peptide, or protein sequence, or a fragment of any of these, and to naturally occurring or synthetic molecules. Where “polypeptide” is recited herein to refer to a polypeptide sequence of a naturally occurring protein molecule, “polypeptide” and like terms are not meant to limit the amino acid sequence to the complete native amino acid sequence associated with the recited protein molecule. The “polypeptide” may be endogenous, exogenous, naturally occurring or recombinant.

The expressions “coding sequence” and “structural sequence” refer to the region of continuous sequential DNA triplets encoding a protein, polypeptide or peptide sequence.

The term “expression” as used herein means the transcription of a gene to produce the corresponding mRNA and translation of this mRNA to produce the corresponding gene product, such as a peptide, polypeptide, or protein.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

The expression “functional fragment” as used herein is intended to mean a nucleotide acid fragment having at least enough molecular elements to confer expression of a recombinant coding sequence.

### BRIEF DESCRIPTION OF DRAWINGS

FIGS. 1A to 1D illustrate the expression of recombinant GFP in Pseudozyma flocculosa;

FIG. 2 illustrates the expression of functional recombinant HEWL in P. flocculosa;

FIG. 3 illustrates the Western blot analysis of recombinant PDGF in P. flocculosa;

FIG. 4 illustrates the expression of functional recombinant PDGF in P. flocculosa by mitogenic activity measurement;

FIGS. 5A to 5D illustrate the expression of recombinant GFP in P. antarctica; and

FIGS. 6A to 6D illustrate the expression of recombinant GFP by single plasmid transformation of P. antartica.

### DESCRIPTION OF PREFERRED EMBODIMENT

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided a method for producing recombinant proteins, peptides and polypeptides through the use of genetically transformed fungi belonging to the genus Pseudozyma. Such genetically transformed Pseudozyma is capable of synthesizing and producing large amounts of heterologous or homologous recombinant gene products. As mentioned herein, no reports in prior art have demonstrated methods of using or simply the use of Pseudozyma spp. as a host for the production of recombinant products.

It has been observed that recombinant proteins may be produced in yields that far exceed those obtainable for the same protein in yeast and that Pseudozyma spp. could synthesize more complex proteins than lower unicellular microorganisms.

In one embodiment of the present invention, the production method makes use, as a host in a host-vector system for the production of recombinant. products, of a strain of Pseudozyma antarctica, Pseudozyma aphidis, Pseudozyma flocculosa, Pseudozyma fusiformata, Pseudozyma prolifica, Pseudozyma rugulosa, Pseudozyma tsukubaensis or any fungal strain which can be identified as a member of the Pseudozyma genus, based on conventional and/or molecular identification techniques.

In a further embodiment of the present invention, there is provided a method for the preparation of a recombinant product that comprises the steps of transforming Pseudozyma protoplasts with a vector containing a gene encoding for the recombinant product and allowing the growth of the transformed strain with a suitable culture media to allow the expression of the recombinant product.

Pseudozyma protoplasts are prepared by removing the cell wall of the fungus, therefore leaving the cytoplasmic membrane as the outermost boundary of the cell. Fungi protoplasts preparation mainly consists in stripping away the cell walls of fungal cells, preferably using enzymes.

Bacterial, yeast, animal, and plant cells have all been transformed. When transforming any type of cell, plasma membranes and/or cell walls must be penetrated without permanently damaging the cell. In the case of fungi, removal of the cell wall is a first and crucial step required before introduction of DNA directly through the plasma membrane. Following protoplast production, the cell membrane can be made permeable to DNA by using electroporation, polyethylene glycol (PEG) or other transformation methods known to the skilled artisan.

One particular embodiment of the present invention is to provide a recombinant expression vector into which a gene of interest is inserted to form a DNA construct. The vector may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated simultaneously with the chromosomes into which it has been integrated.

The gene of interest is inserted into the expression vector as a DNA construct. This DNA construct can be recombinantly made from a synthetic DNA molecule, a genomic DNA molecule, a cDNA molecule or a combination thereof. The DNA construct is preferably made by ligating the different fragments to one another according to standard techniques known in the art.

The gene coding for the recombinant product may be part of the expression vector. Preferably, the expression vector is a DNA vector. The vector conveniently comprises sequences that facilitate the proper expression of the gene of interest. These sequences typically comprise promoter sequences, transcription initiation sites, transcription termination sites, and polyadenylation functions. Additionally, the vector system may comprise a DNA sequence coding for a selection marker. Preferably, this selection marker is capable of being incorporated in the genome of the host organism upon transformation, and was not expressed functionally by the host prior to transformation. Transformed cells can then be selected and isolated from untransformed cells on the basis of the incorporated selection marker.

The promoter sequence is preferably inserted upstream of the gene of interest and regulates its expression. Promoter sequences are non-coding regulatory sequences for transcription, usually located nearby the start of the coding sequence, which may be referred to as the gene promoter or the regulatory sequence. Put into a simplistic yet basically correct way, it is the interplay of the promoter with various specialized proteins called transcription factors that determine whether or not a given coding sequence may be transcribed, and eventually translated into the actual protein encoded by the gene.

It will be recognized by a person skilled in the art that any compatible promoter can be used for recombinant expression in Pseudozyma. For example, but without limiting the scope of the present invention, a promoter originating from other species of fungi or other organisms, can be part of the transcription elements comprised in an expression vector designed for recombinant expression in Pseudozyma, as well as endogenous, synthetic or chimeric promoters.

The promoter itself may be preceded by an upstream activating sequence, an enhancer sequence or combination thereof. These sequences are known in the art as being any DNA sequence exhibiting a strong transcriptional activity in a cell and being derived from a gene encoding an extracellular or intracellular protein.

It will also be recognized by a person skilled in the art that termination and polyadenylation sequences may suitably be derived from the same sources as the promoter.

In one embodiment of the present invention, a target sequence may be inserted in the expression vector to target the protein to a particular cellular localization. Preferably, the target sequence is inserted adjacently to the gene of interest and preserves its open reading frame. Therefore, the gene product is expressed in its entirety but harbors an amino-acid sequence that will direct this gene product through a particular organelle. Although any organelle could be selected within the fungus cell, the extracellular environment is preferred herein, to facilitate the recuperation of the recombinant gene product without damaging producing cells. The target sequence may direct the protein successively to the endoplasmic reticulum, the transfer vesicles, the Golgi apparatus and the secretory vesicles, the latter fusing with the cytoplasmic membrane (exocytosis) to release the protein outside the cell. The release of the protein from the secretory vesicle to the extracellular environment can be constitutive or inducible. The target sequence provided in the vector to ensure efficient direction of the expressed product to the secretory pathway of the host cell may be a naturally occurring signal, leader peptide, a synthetic sequence, a functional part thereof or a combination thereof that provide secretion of the protein from the cell. Thus, the target sequence may be derived from a gene coding for a secreted protein derived from any source including alpha-factor from Saccharomyces sp.

The DNA sequences coding for the recombinant protein, target sequence, promoter and terminator may be inserted into a vector containing a selection marker, or it may be inserted into a separate vector for introduction into the host cell. The vector or vectors may be linear or closed circular molecules. In one embodiment of the present invention, two vectors, one carrying the DNA sequence coding for the selection marker, and the other carrying the DNA sequences encoding the recombinant protein, the target sequence and the functions facilitating gene expression, may be introduced into the host cell.

### EXAMPLES

The present invention will be more readily understood by referring to the following examples which are given to illustrate the invention rather than to limit its scope.

### Example I

### Preparation of Protoplasts of the Fungus Pseudozyma spp

P. flocculosa (DAOM 196992) and P. antarctica (CBS 516.83) were individually cultured in 500 ml Erlenmeyer flasks containing 100 ml yeast malt peptone dextrose broth (YMPDB) at 150 rpm for 3 days at 25° C. Cells (10⁹/ml) were collected by centrifugation, suspended in 20 ml of 25 mM β-mercaptoethanol and 5 mM EDTA pH 8.0, and subjected to gentle shaking for 20 min at room temperature. The cells were then collected and resuspended in various enzyme solutions (see Table 1) prepared in KCE (0.6M KCl, 0.1M sodium citrate and 0.1M EDTA, pH 5.8) and incubated for 2 h at 25° C. The tested enzymes were: Glucanex (5%); Novozym 234 (0.5%, from Trichoderma harzianum); driselase (2%, from basidiomycetes); and Lysing enzymes (5%, from Rhizoctonia solani). All enzymes were obtained from Sigma Chemicals Co. (Toronto, Canada) with the exception of Glucanex which was purchased from Novo Nordisk Ferment Ltd. (Dittingen™, Switzerland). The protoplasts were purified by filtering through a small mass of cotton, and harvested by centrifugation (5000 rpm) for 15 min at 4° C. The protoplasts were then mixed with 10 ml of stabilized yeast malt peptone dextrose agar (YMPDA) containing low-melting-point agar, and immediately spread on a plate containing 10 ml of stabilized YMPDA medium (regeneration medium) for 3 days at 25° C. and regenerated protoplasts were counted as individual colonies.

Results from P. flocculosa protoplast preparation demonstrate that various enzymatic cocktails can be used to efficiently release protoplasts from spores (Table 1).

TABLE 1
Protoplast release from spores of Pseudozyma flocculosa
under different enzymatic treatments

| | Protoplasts (10⁶) of |
|---|---|
| Enzyme * | spores (5 × 10⁷) |
| 5% Glucanex | 4.3 ± 0.4 |
| 0.5% Novozym 234 | 7.7 ± 0.2 |
| 0.5% Novozym 234 ± 5% Glucanex | 11.3 ± 0.9 |
| 0.5% Novozym 234 ± 2% Driselase | 9.5 ± 1.1 |
| 0.5% Novozym 234 ± 5% Lysing | 10.8 ± 0.7 |
| enzymes | |

*Enzyme solutions were prepared in 0.1 M sodium citrate buffer. The yield of protoplasts was obtained after 2 h incubation at 25° C. Results shown represent the means of four replicates.

When protoplasts from both P. flocculosa and P. antarctica were prepared with the Novozym/Glucanex enzymatic cocktails, the protoplast regeneration rate (percentage of protoplasts that form colonies) was ca. 75% for P. flocculosa and ca. 74% for P. antarctica. These results confirm that protoplast quality was suitable for transformation and that, overall, Pseudozyma spp. are useful and efficient hosts for transformation methods through protoplast preparation.

### Example II

### Transformation of Pseudozyma spp.

Protoplasts from P. flocculosa or P antarctica were obtained as described in Example 1. The protoplasts were purified by filtering through cotton, and harvested by centrifugation (5000 rpm) for 15 min at 4° C. To facilitate the transformation procedure, the protoplasts obtained were washed twice with KTC (0.8 M KCl, 25 mM TRIS-HCl pH 8.0 and 50 mM CaCl₂) solution.

Transformation was performed with pScel (donated by Dr. J. Kronstad, University of British Columbia) containing a selection marker gene (hph gene coding for resistance to hygromycin B).

The plasmid was linearized with XhoI and a quantity of 10 μg DNA of each was used to transform the protoplasts. Protoplasts (10⁸) in 200 μl of KTC solution were added to linearized plasmid DNA and incubated in 50 μl of PEG 4000 (66%; BDH, Poole, Dorset, UK) in 50 mM TRIS-HCl pH 8.0, 50 mM CaCl₂ for 20 min at room temperature. An additional 2.5 ml of PEG solution was added in sequential aliquots of 1 drop, 0.5 ml and the remainder (about 2 ml). After another incubation of 20 min at room temperature, the reaction mixture was diluted with 1 ml, 5 ml and 29 ml aliquots of KTC solution. The protoplasts were collected by centrifugation (5000 rpm) for 15 min at 4° C., resuspended in 5 ml YMPD broth containing 0.8 M sucrose as an osmotic stabilizer, and incubated at room temperature for 3 h. The protoplasts were then mixed with 10 ml of stabilized YMPDA (containing low-melting-point agar) medium, and immediately spread on plates containing 10 ml of stabilized YMPDA medium and 100 μg/ml hygromycin. The plates were incubated at 25° C. for 7-14 days.

Colonies from the transformed Pseudozyma cells grew on hygromycin B media whereas the wild-type Pseudozyma did not grow on hygromycin media. Colonies from transformed Pseudozyma colonies were subjected to PCR analysis to confirm the presence of the hygromycin phosphotransferase gene.

Ten putative transformants growing on selective YMPDA media were randomly selected and subjected to PCR analysis to verify the presence of the hph gene. Specific primers reverse (5′-AGCGGATAACAATTTCACACAGGA-3′ SEQ ID NO:1) and hphF (5′-TCGCGGTGAGTTCAGATCTTTTCAT-3′ SEQ ID NO:2) were used to amplify the promoter region and the hph gene of plasmid DNA. Wild-type Pseudozyma on non-selective media was used as a control. PCR was conducted using the following conditions. Initial denaturation was performed at 94° C. for 5 min, followed by 30 cycles of denaturation (94° C., 30 s), annealing (50° C., 30 s), and primer extension (72° C., 1 min), and one final extension (72° C., 10 min).

Agarose gel electrophoresis of the PCR products demonstrated that a band of the expected size was observed in all transformants tested. No PCR product was obtained from the control.

In conclusion, P. flocculosa and P. antarctica are amenable to genetic transformation as PCR analysis confirmed the presence of the hph gene in all tested transformants. Therefore, the genetic transformation is a useful and efficient method for preparing recombinant proteins by way of a host strain of Pseudozyma spp.

### Example III

### Expression of Recombinant Proteins in Pseudozyma spp.

Hygromycin Phosphotransferase

Protoplasts preparation and genetic transformation were carried out as in examples 1 and 2 with P. flocculosa (DAOM 196992) and using transformation with a single plasmid (pCPF.Hyg) containing the hph gene coding for Escherichia coli hygromycin phosphotransferase. Plasmid pCPF.Hyg was constructed by cloning the HSP70 promoter, hph gene, and termination sequences from pScel as an XhoI-SacI fragment into plasmid pBluescript II KS (Stratagene) which was digested by XhoI/SacI with appropriate modification of the protruding ends.

Colonies from the transformed Pseudozyma cells grew on hygromycin B media whereas the wild-type Pseudozyma did not grow on hygromycin media.

Transformants growing on selective YMPDA media were randomly selected and subjected to PCR analysis to verify the presence of the hph gene. Specific primers reverse (5′-AGCGGATAACAATTTCACACAGGA-3′ SEQ ID NO:1) and hphF (5′-TCGCGGTGAGTTCAGATCTTTTCAT-3′ SEQ ID NO:2) were used to amplify the promoter region and the hph gene of plasmid DNA. Wild-type Pseudozyma on non-selective media was used as a control. PCR was conducted using the following conditions. Initial denaturation was performed at 94° C. for 5 min, followed by 30 cycles of denaturation (94° C., 30 s), annealing (50° C., 30 s), and primer extension (72° C., 1 min), and one final extension (72° C., 10 min).

Agarose gel electrophoresis of the PCR products demonstrated that a band of the expected size was observed in all transformants tested. No PCR product was obtained from the control.

In conclusion, hygromycin phosphotransferase was successfully expressed in P. flocculosa as confirmed by PCR analysis and the biological activity of the protein which conferred hygromycin resistance to P. flocculosa.

Green Fluorescent Protein (GFP)

Protoplasts preparation and genetic transformation were carried out as in examples 1 and 2 with P. flocculosa (DAOM 196992) and using co-transformation with plasmid pScel containing the hph gene (selection marker) and another plasmid (pCPF.GFP) containing the GFP gene (S65T) from jellyfish (Aequorea victoria). Plasmid pCPF.GFP was constructed by amplifying the GFP gene from plasmid gGFP (donated by Dr. A. Sharon (Tel Aviv University, Tel Aviv, Israel) using primers gGFP-ClaI.for (5′-CCATCGATATGGCGAGCAAG-3′ SEQ ID NO:3) and gGFP-EcoR.rev (5′-CCGAATTCTCATGTTTGACAG-3′ SEQ ID NO:4). The synthesized fragment containing the GFP gene was digested with ClaI and EcoRI and inserted into pCPF at these sites.

Colonies from the transformed Pseudozyma cells grew on the selection (hygromycin B) media whereas the wild-type Pseudozyma did not grow on hygromycin media. Colonies from transformed Pseudozyma colonies were subjected to fluorescence analysis to confirm the presence of the GFP protein.

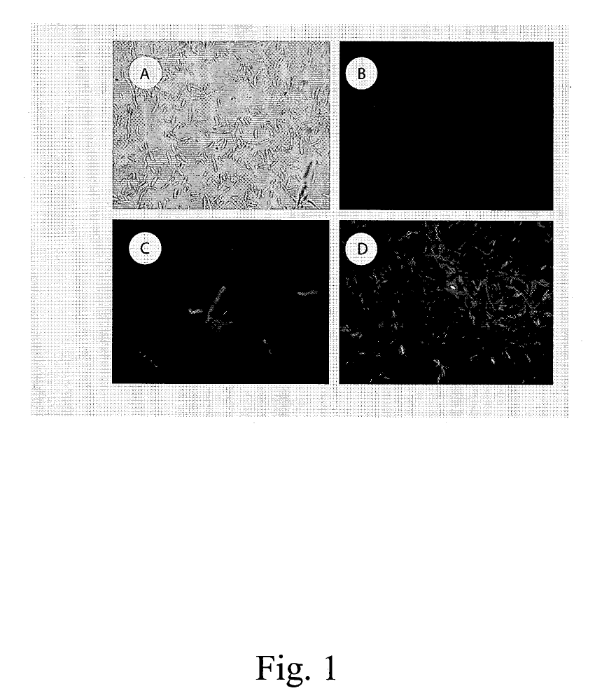

Activity of the GFP protein was observed by fluorescent microscopy at 488 nm of 3-day old cultures of the GFP transformants. These results confirmed that the transformants expressed GFP, in both conidial and mycelial forms, while wild-type P. flocculosa did not demonstrated fluorescence (FIG. 1); P. flocculosa negative control in visible light (A); P. flocculosa as negative control where nothing is being seen at 488 nm (B); transformed P. flocculosa mycelia expressing GFP (C); transformed P. flocculosa conidia expressing GFP (D).

In conclusion, recombinant GFP was successfully expressed in P. flocculosa as confirmed by the fluorescence of the protein in P. flocculosa.

Hen Egg White Lysozyme (HEWL)

Protoplasts preparation and genetic transformation were carried out as in Examples 1 and 2 with of P. flocculosa (DAOM 196992) and using co-transformation with plasmid pScel containing the hph gene (selection marker) and another plasmid (pCPF.HEWL) containing the HEWL gene. Plasmid pCPF.HEWL was constructed by amplifying the HEWL gene from plasmid pSK4-22 (donated by Dr. A. Asselin, Université Laval, Québec, Canada) using primers HEWL-ClaI.for (5′-GGATCGATGAGGTCTTTGCTAATC-3′ SEQ ID NO:7) and HEWL-EcoR.rev (5′-GGGAATTCTCACAGCCGGCAGCCT-3′ SEQ ID NO:8). The synthesized fragment containing the HEWL gene was digested with ClaI and EcoRI and inserted into pCPF at these sites.

Colonies from the transformed Pseudozyma cells grew on the selection (hygromycin B) media whereas the wild-type Pseudozyma did not grow on hygromycin media. Colonies from transformed Pseudozyma colonies were subjected to PCR analysis to confirm the presence of the HEWL gene.

Specific primers HEWL-ClaI.for and HEWL-EcoR.rev were used to amplify the HEWL gene of plasmid DNA. Wild-type Pseudozyma on non-selective media was used as a control. PCR was conducted using following conditions. Initial denaturation was performed at 94° C. for 5 min, followed by 30 cycles of denaturation (94° C., 30 s), annealing (50° C., 30 s), and primer extension (72° C., 1 min), and one final extension (72° C., 10 min).

Agarose gel electrophoresis of the PCR products demonstrated that a band of the expected size was observed in the transformants tested. No PCR product was obtained from the control.

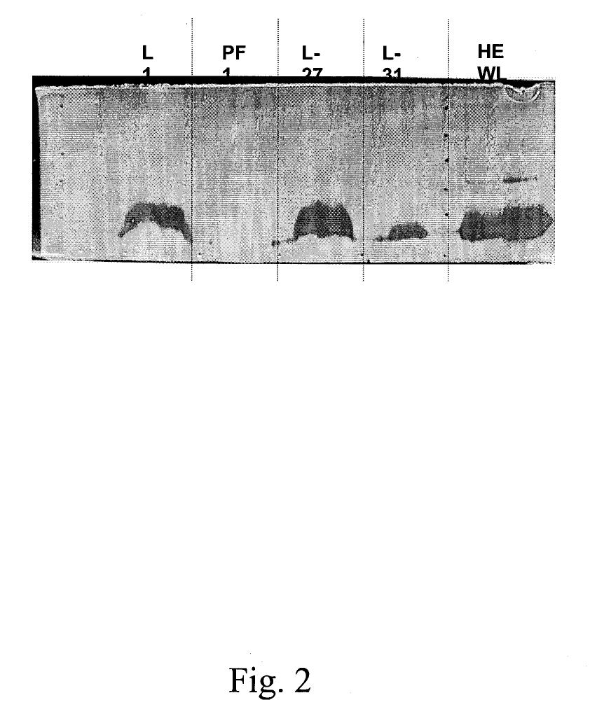

Activity of the HEWL protein was measured lytic activity on electrophoretic gel containing 0.2% autoclaved Micrococcus lysodeikticus cells as previously described (Audy et al., 1989, Comp Biochem Physiol, 92B:523-527). Lysozyme activity is revealed by lysis zones on dark background. These results confirmed that the transformants expressed the functional HEWL protein in the tested transformants, whereas wild-type P. flocculosa did not demonstrate HEWL activity (FIG. 2); L-1, L-27, and L-31: three P. flocculosa transformants expressing functional HEWL; PF 1: P. flocculosa control; HEWL: purified Hen Egg White Lysozyme.

In conclusion, recombinant HEWL was successfully expressed in P. flocculosa as confirmed by PCR analysis and the biological (lytic) activity of the protein from P. flocculosa.

Human Platelet-Derived Growth Factor (PDGF)

Protoplasts preparation and genetic transformation were carried out as in examples 1 and 2 with P. flocculosa (DAOM 196992) and using co-transformation with plasmid pScel containing the hph gene (selection marker) and another plasmid (pCPF.PDGF) containing the human PDGF gene. Plasmid pCPF.PDGF was constructed as follows:

The PDGF gene was obtained as a plasmid constructed by amplifying the gene contained on plasmid pSM-1 (ATCC 57050) which was inserted into the vector pET11a (Novagen) by the sites NdeI and BamHI. For transformation into P. flocculosa, the PDGF gene was obtained by amplifying the PDGF gene from this construct using primers PDGF-ClaI.for (5′-GTTTATCGATAAGAAGGAGATATAC-3′SEQ ID NO:7) and PDGF-EcoR.rev (5′-GTTAGCAGCCGAATTCCTATTAGGT-3′ SEQ ID NO:8). The synthesized fragment containing the PDGF gene was digested with ClaI and EcoRI and inserted into pCPF at these sites.

Colonies from the transformed Pseudozyma cells grew on the selection (hygromycin B) media whereas the wild-type Pseudozyma did not grow on hygromycin media. Colonies from transformed Pseudozyma colonies were subjected to PCR analysis to confirm the presence of the PDGF gene.

Specific primers PBS (5′-CAAGCGCGCAATTAACCCTCACTA-3′SEQ ID NO:9) and For.terminator (5′-CACCACCTACTCACGACTGTTG-3′ SEQ ID NO:10) were used to amplify the PDGF gene of plasmid DNA. Wild-type Pseudozyma on non-selective media was used as a control. PCR was conducted using following conditions. Initial denaturation was performed at 94° C. for 5 min, followed by 30 cycles of denaturation (94° C., 30 s), annealing (50° C., 30 s), and primer extension (72° C., 1 min), and one final extension (72° C., 10 min).

Agarose gel electrophoresis of the PCR products demonstrated that a band of the expected size was observed in the transformants tested. No PCR product was obtained from the control.

ELISA analysis of the transformants was carried out with anti-hPDGF-bb antibody (R&D systems, #AB-220-NA) in order to confirm the presence of PDGF. The antibody recognizes both the inactive monomer (PDGF-B) and the active dimer (PDGF-BB).

Protein extracts of PDGF transformants were performed on 70-hr old liquid cultures in YMPD broth. Wild-type P. flocculosa served as the control. The fungal cells were separated from the culture medium by centrifugation (21,000×g for 20 min). The culture medium was lyophilized and resuspended in YBB buffer (Qbiogen, Carlsbad, Calif.) with an addition of a 1/10 dilution of general use protease inhibitor cocktail (Sigma-Aldrich, Oakville, Ontario, Canada). The fungal pellet was submitted to soluble protein extraction using the FastProtein™ Matrix system (Qbiogen, Carlsbad, Calif.) according to the manufacturer's specifications in YBB buffer with an addition of a 1/10 dilution of general use protease inhibitor cocktail.

ELISA analysis was carried out on both the culture media and the soluble protein fractions using the anti-hPDGF-bb antibody (R&D systems, #AB-220-NA) according to the manufacturer's specifications using Anti-Goat IgG alkaline phosphatase (Sigma). The presence of PDGF was revealed on an ELISA plate reader (Multiskan Ascent Labsystems) at 405 nm using purified PDGF-B and PDGF-BB as positive controls.

Results demonstrated that PDGF was expressed in the soluble protein fraction of transformants as well as in some of the culture media and that the wild-type fungus did not express PDGF.

In order to identify the form of the PDGF protein expressed in P. flocculosa, Western blot analysis was performed on the transfonnant showing the highest degree of expression in ELISA analysis as well as the wild-type fungus. The transformant and wild-type fungus were cultured in 50 ml YMPD broth for 72 hrs at 25° C. (150 rpm). The culture media was centrifuged (15,000×g for 10 min) and the fungal cell pellet was suspended in 25 mL of PBS buffer. Cells were lysed by two passages in a French R pressure cell press (SLM Aminco Instruments Inc., Urbana, Ill.) set at a pressure of 14000-16000 psi. The lysate was centrifuged at 15,000×g, at 4° C., for 20 min. The pellet was suspended in 10 mL TE+buffer (10 mM Tris pH 8.0, 10 mM EDTA pH 8.0). 2 μg of the pellet and the soluble (supernatant) fraction were deposited on 15% reducing SDS-PAGE gel. Following electrophoresis separation, the proteins were transferred to a nitrocellulose membrane. The membrane was blocked for one hour with 5% milk in PBS containing 0.05% Tween 20. The membrane was incubated for 1 hr with anti-hPDGF-BB diluted 1/1000 in 0.5% milk in PBS containing 0.05% Tween 20™ and washed three times for five minutes with PBS-Tween 20. The membrane was then incubated for 30 min with a 1/10,000 anti-goat peroxydase (Jackson ImmunoResearch, #705-035-003) and washed three times for eight min in PBS-Tween 20. The presence of PDGF was revealed by chemiluminescence.

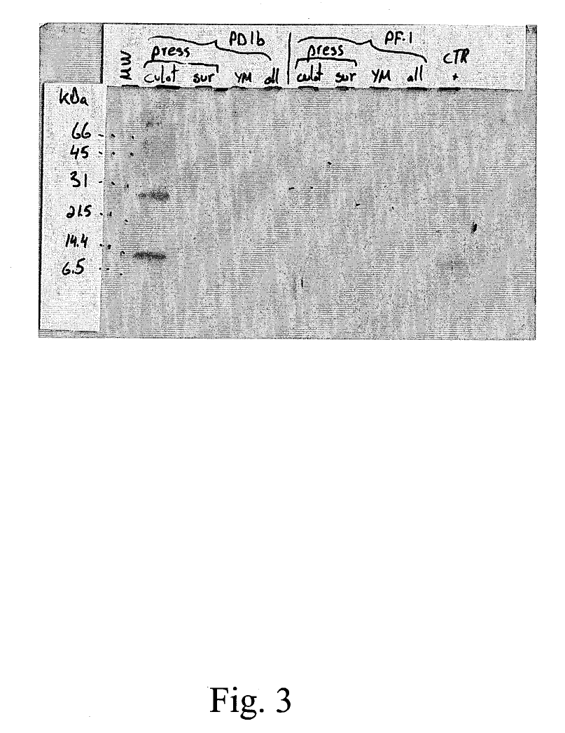

Western blot analysis of the transfonnant and the wild-type fungus confirmed that the transformant expressed not only the 14 kDa monomer PDGF-B but also the 28 kDa dimer PDGF-BB, even in reducing conditions, and that the protein almost exclusively retrieved in the fungal pellet (FIG. 3); MW: Molecular weight marker; PD1b: transformed P. flocculosa expressing PDGF; PF-1; P. flocculosa control; Culot: protein extract from fungal pellet; Sur: soluble protein extract; YM: culture media extract; All: culture media+soluble protein extract; CTR; PDGF-B control.

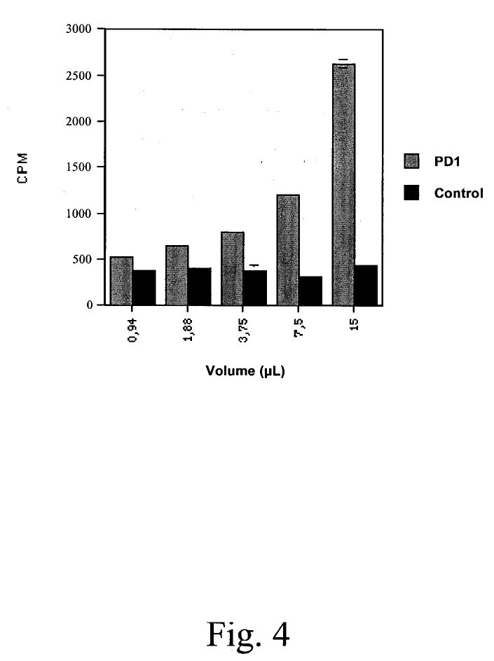

Activity of the PDGF protein was measured by in vitro mitogenic activity in rat kidney cells by (³H)-thymidine incorporation (CPM) as previously described (Lariviére et al., 2003, Wound Rep Reg, 11:79-89). The assay revealed measurable mitogenic activity in the transformant whereas no activity was observed in the wild-type fungus treatment and the untreated cells confirming the presence of the bioactive dimer (PDGF-BB; FIG. 4); PD1: transformed P. flocculosa expressing PDGF activity; Control: Non-treated cells.

In conclusion, PDGF was successfully expressed in P. flocculosa in its active dimer form as confirmed by PCR analysis, ELISA, Western blot assay, and the biological (mitogenic) activity of the recombinant protein from P. flocculosa.

### Example IV

### Expression of GFP with Various Pseudozyma Species

In addition to expression in P. flocculosa, GFP was expressed in P. antarctica (CBS 516.83) using methods identical to that of Example 3.

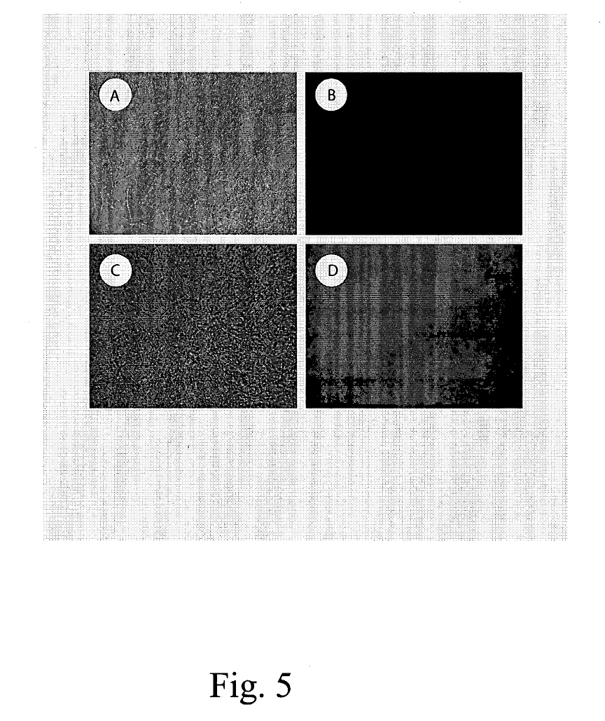

These results confirmed that the transformants expressed GFP while wild-type P. antarctica did not demonstrate fluorescence (FIG. 5); P. antarctica as negative control in visible light (A); P. antarctica as negative control at 488 nm (B); transformed P. antarctica in visible light (C); transformed P. antarctica at 488 nm (D).

In conclusion, recombinant GFP was successfully expressed in P. antarctica as confirmed by the fluorescence of the protein.

Overall, the results confirm that various species of Pseudozyma are useful as hosts for the expression of recombinant products.

### Example V

### Expression of GFP with Various Vectors

In addition to co-transformation of P. antarctica with plasmids pScel and pCPF.GFP, GFP was expressed in P. antarctica using a single plasmid pSPF.GFP as follows.

Protoplasts preparation and genetic transformation were carried out as in Examples 1 and 2 with P. antarctica (CBS 516.83) using single plasmid transformation with plasmid pSPF.GFP containing the hph gene (selection marker) and the GFP gene (S65T) from jellyfish (Aequorea Victoria). Plasmid pSPF.GFP was constructed by amplifying the hsp70 promoter, the GFP gene, and the hsp70 terminator sequences from plasmid pCPF.GFP using primers KpnI-pro-gGFP (5′-GAGTGGTACCAGATGTGAGTCGT-3′ SEQ ID NO:11) and SacI-ter-gGFP (5′-GGAGCTCGATAACCGGGATCCG-3′ SEQ ID NO;12). The fragment obtained was digested with KpnI and SacI and was inserted between the KpnI and SacI sites of pScel-Hyg to obtain the expression vector pSPF.GFP.

Colonies from the transformed Pseudozyma cells grew on the selection (hygromycin B) media whereas the wild-type Pseudozyma did not grow on hygromycin media. Colonies from transformed Pseudozyma colonies were subjected to analysis to confirm the presence of the GFP protein.

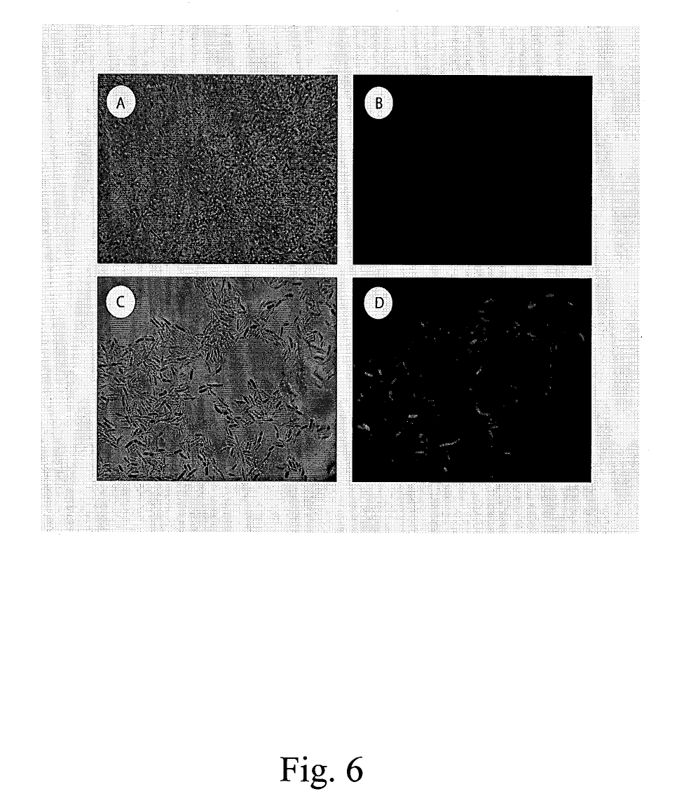

Activity of the GFP protein was observed by fluorescent microscopy at 488 nm of 3-day old cultures of the GFP transformants. These results confirmed that the transformants expressed GFP while wild-type P. antarctica did not demonstrate fluorescence (FIG. 6); P. antarctica as negative control in visible light (A); P. antarctica as negative control at 488 nm (B); transformed P. antarctica in visible light (C), and transformed P. antarctica at 488 nm (D).

In conclusion, recombinant GFP was successfully expressed in P. antarctica as confirmed by the fluorescence of the protein in P. antarctica.

Overall, these results confirm that various vectors/transformation methods can be used for the expression of recombinant products in Pseudozyma spp.

### Example VI

### Expression of Recombinant Proteins with Various Regulatory Sequences

HSP70 Truncated Promoter

Successive 100-bp deletions from the 5′-end of the HSP70 promoter were carried out as follows.

Amplification, using pScel as the template, was carried out using the following forward primers: HSP70-100.for (5′-GCCTCGAGATTTGTTCCAAGTTCAAA-3′ SEQ ID NO:13), HSP70-200.for (5′-AGCTCGAGGAAGAAGAACGTGGTAAC-3′ SEQ ID NO:14), HSP70-300.for (5′-ATCTCGAGGGCTAAAGGAAGCGAGAC-3′ SEQ ID NO:15), HSP70-400.for (5′-ATCTCGAGTTGCGTCAGCCTTGTACC-3′ SEQ ID NO:16), HSP70-500.for (5′-ATCTCGAGATGACACAGCACACCAAG-3′ SEQ ID NO:17). PCR was carried out using one of the forward primers and primer antipro-all.rev (5′-ATCCATGGCCTCCGCGACCGGCTGCAGAA-3′ SEQ ID NO:18) to obtain the five versions of the deleted HSP70 promoter.

The five amplified DNA sequences were digested with XhoI and NcoI and inserted at this site on plasmid pSceI. The resulting plasmids individually contained successive 100-bp deletions of the HSP70 promoter driving the hph gene coding for hygromycin phosphotransferase.

Protoplast preparation and transformation of P. antarctica was carried out as in examples 1 and 2 with the five plasmids containing the truncated versions of the HSP70 promoter as well as the plasmid containing the original full length promoter (pSceI).

Results demonstrated that all versions of the promoter allowed the expression of hygromycin phosphotransferase as witnessed by hygromycin B resistance in the transformed fungi (Table 2).

TABLE 2
Expression of hygromycin phosphotransferase by P. antarctica
transformed with vectors containing truncated HSP70 promoters

| Promoter | Number of transformants expressing hph* |
|---|---|
| None (control) | 0 |
| Full length HSP70 | ca. 10E7 |
| 100 bp deletion | ca. 10E4-10E5 |
| 200 bp deletion | ca. 10E3-10E4 |
| 300 bp deletion | 2.4X10E2 |
| 400 bp deletion | 29 |
| 500 bp deletion | 6 |

*hph = hygromycin phosphotransferase gene conferring resistance to hygromycin B. Values are means of 3 replicates.

These results confirm that various promoter sequences can be used successfully to express proteins in Pseudozyma spp.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A method for the production of a recombinant polypeptide in fungus Pseudozyma spp. comprising the steps of:
a) genetically transforming a strain of Pseudozyma with an expression vector; and
b) cultivating said transformed strain of Pseudozyma of step a) under conditions allowing synthesis of a recombinant polypeptide from said expression vector.

2. The method of claim 1, wherein said transforming of step a) is a genetic transformation of protoplasts.

3. The method of claim 1, wherein said strain of Pseudozyma is Pseudozyma antarctica, Pseudozyma aphidis, Pseudozyma flocculosa, Pseudozyma fusiformata, Pseudozyma prolifica, Pseudozyma rugulosa or Pseudozyma tsukubaensis.

4. The method of claim 1, wherein said expression vector is a plasmid, an expression cassette, or a compatible virus.

5. The method of claim 1, wherein said expression vector comprises a DNA sequence encoding a recombinant protein or peptide, and a promoter active in the genus Pseudozyma.

6. The method of claim 4, wherein said expression vector comprises a preregion allowing secretion of an expressed recombinant protein or peptide.

7. The method of claim 4, wherein said promoter is native to a strain of Pseudozyma.

8. The method of claim 4, wherein said promoter is an ubiquitous or an inducible promoter.

9. A fungus of the genus Pseudozyma genetically transformed for production of a recombinant polypeptide.

10. Use of a Pseudozyma spp. fungal strain as a host in a host-vector system for the production of recombinant proteins and other recombinant polypeptides.

11. A composition comprising a genetically transformed fungus of the genus Pseudozyma for production of a recombinant polypeptide
